(19) United States
(12) Patent Application Publication
(10) Pub. No.: US 2006/0165632 A1
(43) Pub. Date: Jul. 27, 2006

(54) Use of carbohydrates to improve skin barrier function

(75) Inventor: Bruno Mehul, Villejuif (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: SOCIETE L'OREAL S.A., Paris (FR)

(21) Appl. No.: 11/200,243

(22) Filed: Aug. 10, 2005

Related U.S. Application Data
(63) Continuation of application No. 10/436,155, filed on May 13, 2003, now abandoned.
Application No. 10/436,155 is a continuation of application No. PCT/FR01/03550, filed on Nov. 13, 2001.

(30) Foreign Application Priority Data
Nov. 13, 2000 (FR) FR-00/14557

(51) Int. Cl. C12Q 1/34; A61K 8/73

(52) U.S. Cl. 424/70.13; 435/18

(57) ABSTRACT

The present invention concerns compositions containing carbohydrate derivatives for enhancing the skin barrier function. The invention further concerns a cosmetic treatment method for enhancing the skin barrier function by applying on the skin compositions comprising carbohydrate derivatives.

### CROSS-REFERENCE TO RELATED APPLICATIONS

This application is a continuation of U.S. application Ser. No. 10/436,155, filed May 13, 2003, which is a continuation of PCT/FR01/03550, filed Nov. 13, 2001, which claims priority under 35 U.S.C. §119 of FR-00/14557, filed Nov. 13, 2000, all of the prior applications being expressly incorporated by reference herein in their entireties and relied upon.

The invention relates to the use of carbohydrate derivatives in a composition for improving skin barrier function. It also relates to a cosmetic treatment method for improving the skin barrier function by applying compositions comprising carbohydrate derivatives to the skin.

Human skin is constituted by two compartments, namely a deep compartment, the dermis, and a superficial compartment, the epidermis.

The epidermis is in contact with the external environment. It protects the organism from dehydration and from external chemical, mechanical or infectious attack.

The cells constituting the epidermis are defined by an intercellular lipid structure. During differentiation, phospholipids, which produce the fluid structure of the cell membranes in the living layers of the epidermis, are gradually replaced by a mixture mainly composed of fatty acids, cholesterol and sphingolipids. Those lipids are organized into specific lamellar structures the integrity of which depends not only on the quality of the fractions present, but also on their respective proportions. That lamellar lipid structure is responsible for the suppleness of the skin. Of the lipids, sphingolipids (ceramides) are essential for maintaining the multilamellar structure of intercomeocyte lipids. They are essential to water exchanges and to the “barrier” function of the epidermis.

Inter-corneocyte lipids undergo modifications. This maturation is necessary for establishing a proper barrier function. Deglycosilation of lipid precursors such as glycosylceramide to ceramide is modulated by the action of specific endogenous glycosidases (glucosidases). As a result, that deglycosilation is an important step in developing the skin barrier function.

The lipids of the skin, in particular the epidermis, are influenced by genetic factors, aging, diet, environmental factors, attack and/or certain diseases (scurvy or pellagra, for example). Those factors alter or modify the composition of the lipids in the skin or reduce the quantity, resulting in dry skin.

The invention results from in vitro and in vivo studies of the effect of carbohydrate derivatives on the skin.

One use of said carbohydrate derivatives to encourage skin desquamation has already been described in L'Oréal's International patent application “Use of carbohydrate to encourage skin desquamation” (WO-A-97/12597).

We have now, surprisingly, discovered a novel effect of carbohydrate derivatives, obtained by specific stimulation of certain β-glucosidases which manifests itself in an improvement in the skin barrier function and/or the mucous membrane barrier function, particularly when the carbohydrate derivatives are applied topically.

In other words, the invention concerns compositions used to improve the skin barrier function; said function can be correlated to β-glucosidase activity, such that the improvement in the barrier function can be revealed by a stimulation in β-glucosidase activity.

Particular β-glucosidases that are involved in glycolipid catabolism are glucosyl-ceramides. A specific increase in β-glucosidase activity can increase the amount of ceramides in skin lipids, thereby improving the skin barrier function.

In particular, O-octanoyl-6′-maltose has been shown to have an effect on stimulating the activity of certain glycosidases, more particularly β-D-glucosidase in the stratum corneum. Further, that effect is correlated in vivo to a substantial increase in the skin barrier function.

Thus, the invention provides, in a composition for improving the skin barrier function, at least one carbohydrate or carbohydrate derivative with general formula (I):
R—X-A (I)
in which A represents a chain composed of one to twenty carbohydrate units or carbohydrate derivatives each containing 3 to 6 carbon atoms, connected together, preferably via acetal bridges, each of said units possibly being substituted, for example with a halogen, an amine function, an acid function, an ester function, a thiol, an alkoxy function, a thio-ether function, a thio-ester function, an amide function, a carbamate function or a urea function;
R represents a linear or branched alkyl chain or an alkenyl chain, containing 1 to 24, preferably 4 to 24 carbon atoms, which can be interrupted by ether bridges, optionally carrying a hydroxyl function, a carboxylic acid function, an amine function, an ester function, an acyloxy function, an amide function, an ether function, a carbamate function, or a urea function;
X represents a function connecting R and A, such as an amine, ether, amide, ester, urea, carbamate, thioester, thioether or sulphonamide function.

Preferably, R represents a linear or branched alkyl chain or an alkenyl chain containing 4 to 24 carbon atoms, optionally carrying a hydroxyl function.

Each of the carbohydrate component A units can be a sugar or a sugar derivative. As an example, each component A unit can be a reduced sugar, an amino sugar or a sugar carrying a carboxylic acid function.

Examples of sugars or sugar derivatives that can form part of A which can be cited are the following commercially available products, possibly in their salt form: N-acetyl-D-galactosamine, N-acetyl-D-glucosamine, N-acetyl-neuraminic acid, adonitol, β-D-allose, β-D-altrose, 6-amino-6-deoxy-D-glucose, 1,6-anhydroglucose, arabinic acid, arabinogalactan, D-arabinose, L-arabinose, D,L-arabinose, D-arabitol, D-cellobiose, D-glucosamine, D-galactosamine, 2-deoxy-D-glucose, 6-deoxy-D-galactose, 6-deoxy-L-galactose, galactitol, mesoerythritol, D-erythrose, D-fructose, D-fucose, L-fucose, D-galactaric acid, galactitol, galactomannane, D-galactono-1,4-lactone, L-galactono-1,4-lactone, D-galactosamine, D-galactose, L-galactose, D-galacturonic acid, β-gentobiose, glucamine, D-glucaric acid, D-glucono-1,5-lactone, L-glucono-1,5-lactone, D-glucosamine, D-glucosaminic acid, D-glucoronic acid, L-glucose, D-glucose, isomaltitol, isomaltotriose, isomaltose, lactobionic acid, lactulose, D-lyxose, L-lyxose, lyxosamine, maltitol, D-maltose, maltotetraose, maltotriitol, maltotriose, D-mannosamine, D-mannose, L-mannose, D-melezitose, D-melibiose, D-raffinose, D-raffinose undeca-acetate, L-rhamnose, D-ribose, L-ribose, D-ribulose, rutinose, D-saccharose, a-sophorose, sorbitol, D-tagatose, D-talose, D-threose, turanose, D-xylitol, D-xylose, L-xylose, D,L-xylose.

Preferably, A is selected from the following hydrocarbon chains:

D-glucosamine or 2-amino-2-deoxy-D-glucose, D-glucaamine or 1-amino-1-deoxy-D-glucitol, N-methylglucamine, D-glucose, D-maltose, sorbitol, maltitol.

Preferably, R contains 4 to 16 carbon atoms, such as an n-butyl, n-octyl, 2-ethylhexyl or n-dodecyl radical.

In accordance with the invention, preferred compositions comprise at least one product selected from:

N-butanoyl-D-glucosamine, N-octanoyl-D-glucosarnine, N-octyloxycarbonyl-N-methyl-D-glucamine, N-2-ethylhexyloxycarbonyl-N-methyl-D-glucamine, 6′-O-octanoyl-D-maltose, 6′-O-dodecanoyl-D-maltose.

The preparation of products (I) is well known to the skilled person. In this regard, reference should be made, for example, to the following French and European patents: FR-A-2 703 993, FR-A-2 715 933, EP-A-0 577 506, EP-A-0 566 438 and EP-A-0 485 251.

More preferably, a carbohydrate derivative with formula (I) is O-octanoyl-6′-maltose.

In the compositions of the invention, the carbohydrate (I) or the mixture of carbohydrates (I) can be used in a quantity of 0.05% to 20% by weight with respect to the total composition weight, in particular in a quantity of 0.2% to 10%, preferably 0.5% to 5% by weight with respect to the total composition weight.

The carbohydrates (I) can be selected by means of an in vitro test described in the experimental section (part 1.1).

As a result, the invention pertains to the use of compositions as described above, wherein the carbohydrate derivative is selected using an in vitro test that can quantify the stimulant effect of said derivative on the β-D-glucosidase activity, said test comprising the following steps:
a) producing a mixture constituted by the carbohydrate derivative, a β-D-glucosidase, a chromogenic substrate for said β-D-glucosidase, and a suitable buffer solution;
b) quantifying the rate of the enzymatic β-glucosidase reaction, in particular by assaying the quantity of chromophores released by cleavage of the chromogenic substrate; and
c) selecting carbohydrate derivatives for which the reaction rate is improved compared with the rate of reaction measured in a control solution in the absence of derivatives.

The carbohydrate derivatives are used in accordance with the invention in a composition containing a cosmetically or dermatologically acceptable medium, i.e., a medium that is compatible with the skin, nails and mucous membranes, the tissues and the skin. In a preferred embodiment of the invention, the pH of the composition is close to that of skin, in the range 4 to 7. When applied topically, the composition comprising one or more carbohydrate derivatives can be applied to the skin, the neck, the hair, the mucous membranes and the nails or any other cutaneous area of the body.

The composition is preferably in a form that is suitable for administration by topical application. It is usually in the form of hydroalcoholic or oily solutions, lotion or serum type dispersions, anhydrous or oily gels, milk type emulsions with a liquid or semi-liquid consistency obtained by dispersing an oily phase in an aqueous phase (O/W) or vice versa (W/O), suspensions or emulsions with a soft, semi-solid or solid consistency of the cream, gel or micro-emulsion type, or as micro-capsules, micro-particles, or ionic and/or non ionic type vesicular dispersions. Said compositions are prepared using the usual methods.

The compositions of the invention can also be used for the hair in the form of alcoholic or hydroalcholic solutions, or in the form of creams, gels, emulsions or foams.

The quantities of the different constituents of the compositions used in accordance with the invention are those that are routinely used in the fields under consideration.

Said compositions constitute creams for protection, treatment or care of the face, hands or body, milks for protecting or caring for the body, lotions, gels or foams for care of the skin and mucous membranes, or for cleaning the skin.

The compositions can also consist of solid preparations constituting soaps or cleansing bars.

In known manner, the composition of the invention can also contain adjuvants that are in normal use in the cosmetic and dermatological fields, such as hydrophilic or lipophilic gelling agents, hydrophilic or lipophilic active ingredients, preservatives, antioxidants, solvents, fragrances, fillers and colorants. The quantities of said adjuvants are those that are conventionally used in the fields under consideration, for example 0.01% to 20% of the total composition weight. Clearly, the skilled person will be careful to select any additives and/or their quantities so that the advantageous intrinsic properties of the composition of the invention are not or are not substantially altered by the envisaged adjuvants.

Oils that can be used in the invention that can be cited are mineral oils (Vaseline oil), vegetable oils (shea oil, sweet almond oil), animal oils, synthesized oils, silicone oils (cyclomethicone), and fluorinated oils (perfluoropolyethers). It is also possible to use fatty alcohols, fatty acids (stearic acid) or waxes (paraffin, camauba, beeswax) as the oily materials.

Emulsifying agents that can be used in the invention that can be cited are polysorbate 60 and sorbitan stearate sold by ICI under the respective trade names of Tween 60 and Span 60. Co-emulsifying agents can be added, such as PPG-3 myristyl ether sold by Witco as Emcol 249-3K.

Solvents that can be used in the invention that can be cited are lower alcohols, in particular ethanol and isopropanol, and propylene glycol.

Hydrophilic gelling agents that can be cited are carboxyvinyl polymers (carbomers), acrylic copolymers such as acrylate/alkylacrylate copolymers, polyacrylamides, polysaccharides such as hydroxypropylcellulose, natural gums (xanthan), and clays; lipophilic gelling agents that can be cited are modified clays such as bentonites, metallic sols of fatty acids such as aluminium stearates, hydrophobic silica, polyethylenes and ethylcellulose.

Hydrophilic active ingredients that can be used include proteins or protein hydrolysates, amino acids, polyols, urea, allantoin, sugars and sugar derivatives, hydrosoluble vitamins, starch, or bacterial or vegetable extracts, in particular aloe vera.

Lipophilic active ingredients that can be used include tocopherol (vitamin E) and its derivatives, essential fatty acids, ceramides and essential oils.

In order to combat photoaging effectively, it is also possible to add to the composition of the invention one or more complementary sunscreens that are active in the UVA and/or UVB, which may be hydrophilic or hydrophobic, optionally including a sulphonic function. The sunscreen is preferably selected from organic and/or mineral sunscreens.

Organic sunscreens that can be cited are cinnamic derivatives, salicylic derivatives, camphor derivatives, triazine derivatives, benzophenone derivatives, dibenzoylmethane derivatives, β,β-diphenylacrylate derivatives, p-aminobenzoic acid derivatives, polymeric sunscreens and silicone sunscreens described in patent application WO-A-93/04665, or organic sunscreens described in patent application EP-A-0 487 404.

Mineral sunscreens that can be cited are pigments, or preferably nanopigments (mean primary particle size: generally in the range 5 nm to 10 nm, preferably in the range 10 nm to 50 nm) of coated or uncoated metal oxides, such as nanopigments of titanium oxide (amorphous or crystalline in the form of rutile and/or anatase), iron oxide, zinc oxide, zirconium oxide or cerium oxide, which are all well known photoprotective agents acting by physically blocking (reflection and/or diffusion) UV radiation. Alumina and/or aluminium stearate are conventional coating agents. Such coated or uncoated metal oxide nanopigments have in particular been described in patent applications EP-A-0 518 772 and EP-A-0 518 773.

Examples of complementary sunscreens that are active in the UV-A and/or UV-B region that can be cited are:
p-aminobenzoic acid;
oxyethylenated (25 mol) p-aminobenzoate;
2-ethylhexyl p-dimethylaminobenzoate;
N-oxypropylenated ethyl p-aminobenzoate;
glycerol p-aminobenzoate;
homomenthyl salicylate;
2-ethylhexyl salicylate;
triethanolamine salicylate;
4-isopropylbenzyl salicylate;
4-tert-butyl-4′-methoxy-dibenzoylmethane (PARSOL 1789 from GIVAUDAN ROURE);
2-ethylhexyl p-methoxycinnamate (PARSOL MCX from GIVAUDAN ROURE);
4-isopropyl-dibenzoylmethane (EUSOLEX 8020 from MERCK);
menthyl anthranilate;
2-ethylhexyl-2-cyano-3,3′-diphenylacrylate (UVINU N539 from BASF);
ethyl-2-cyano-3,3′-diphenylacrylate;

2-phenyl benzimidazole 5-sulphonic acid and its salts;
3-(4′-trimethylammoniun)-benzylidene-boman-2-one-methylsulphate;
2-hydroxy-4-methoxybenzophenone (INUL MS 40 from BASF);
2-hydroxy-4-methoxybenzophenone-5-sulphonate (UVINUL MS 40 from BASF);
2,4-dihydroxybenzophenone (UVINUL 400 from BASF);
2,2′,4,4′-tetrahydroxybenzophenone (IVNUL D 50 from BASF);
2,2′-dihydroxy-4,4′-dimethoxybenzophenone (HELOSORB II from NORQUAY);
2-hydroxy-4-n-octoxybenzophenone;
2-hydroxy-4-methoxy-4′-methylbenzophenone;
α-(2-oxobom-3-ylidiene)-tolyl-4-sulphonic acid and its salts;
3-(4′-sulpho)benzylidene-boman-2-one and its salts;
3-(4′-methylbenzylidene)-d,1-camphor;
3-benzylidene-d,1-camphor;
benzene 1,4-di(3-methylidene-10-camphorsulphonic) acid and its salts (MEXORYL SX from CHIMEX);
urocanic acid;
2,4,6-tris-[p-(2′-ethylhexyl-1′-oxycarbonyl)anilino]-1,3,5-triazine;
2-[p-(tertiobutylamido)anilino]-4,6-bis[p-(2′-ethylhexyl-1′-oxycarbonyl)anilino]-1,3,5-triazine;
2,4-bis{[4-2-ethylhexyloxyl]-2-hydroxyphenyl}-6-(4-methoxyphenyl)-1,3,5-triazine;
the polymer of N-(2 and 4)-[2-oxoborn-3-ylidene)methyl)benzyl]-acrylamide;
4,4-bis-benzimidazolyl-phenylene-3,3′,5,5′-tetrasulphonic acid and its salts;
2,2′-methylene-bis-[6-(2H-benzotriazole-2-yl)-4-(1,1,3,3-tetramethylbutyl)phenol];
polyorganosiloxanes with a malonate function.

The invention also concerns a cosmetic treatment method implemented by applying compositions as defined above using the normal technique for using said compositions, said cosmetic method improving the skin barrier function. Examples are: application of creams, gels, serums, ointments, lotions, milks to the skin, the scalp, the nails and/or the mucous membranes.

The experimental section gives the results obtained of in vitro and in vivo studies on the effect of an example of at least one carbohydrate derivative. Non-limiting examples of compositions for use in accordance with the invention to improve the skin barrier function are also given.

### Experimental Section

### DESCRIPTION OF FIGURES

FIG. 1: FIG. 1 is a graph showing the effect of O-octanoyl-6′-maltose on glycosidases of the stratum corneum. The activity is expressed as the percentage activity with respect to the value measured in the absence of O-octanoyl-6′-maltose.

FIG. 2: FIG. 2 illustrates the effect of different concentrations of O-octanoyl-6′-maltose, O-octanoyl-6′-glucose, maltose and glucose on β-glucosidase activity.

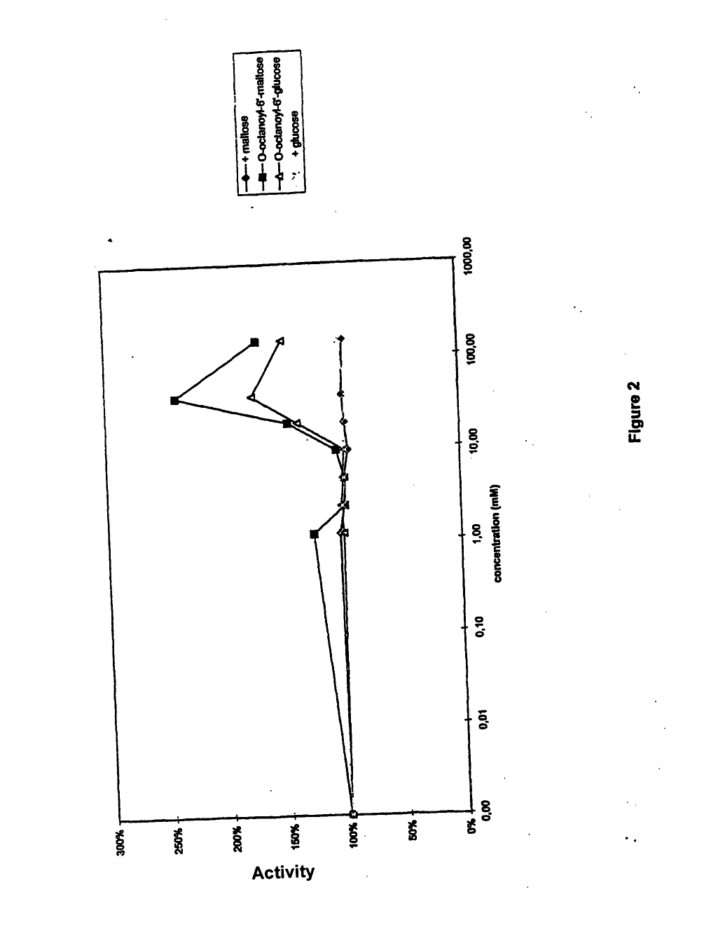

FIG. 3: FIG. 3 illustrates the effect of O-octanoyl-6′-maltose on each test recombinant glucosidase (CloneEnzyme).

### 1 STUDY OF A CARBOHYDRATE DERIVATIVE, O-OCTANOYL-6′-MALTOSE [α-D-GLUCOPYRANOSYL-1-4-D-GLUCOPYRANOSE] ON GLYCOSIDASE ACTIVITY STIMULATION

### 1.1 Effect of O-octanoyl-6′-maltose on the Activity of Glycosidases in the Stratum Corneum

In order to determine the nature and importance of the barrier effect of a carbohydrate derivative in accordance with the invention, the influence of O-octanoyl-6′-maltose on glycosidase activity was studied using the following enzymatic test:

The assay was carried out in a 96 well plate. A mixture comprising the following was produced:
75 μl of a 10 mM solution of specific chromogenic substrate coupled to para-nitrophenol (PNP);
10 μl of 700 mM citrate buffer/200 mM sodium phosphate, pH 4.5;
55 μl of a 0.5 M solution of O-octanoyl-6′-maltose [α-D-glucopyannosyl-1 -4-D-glucopyranose];
40 μl of a solution of soluble glycosidases from the stratum comeum. This solution was obtained simply by scratching the forearm into a 70 mM sodium citrate buffer, pH 4.5 containing 1% of Tween 20 and eliminating cell debris by sequential filtering through a membrane with a pore size of 0.45 and of 0.22 μm.

The measurements were carried out by quantifying the yellow coloration after incubation at 37° C. for a period in the range 1h30 and 48 hours.

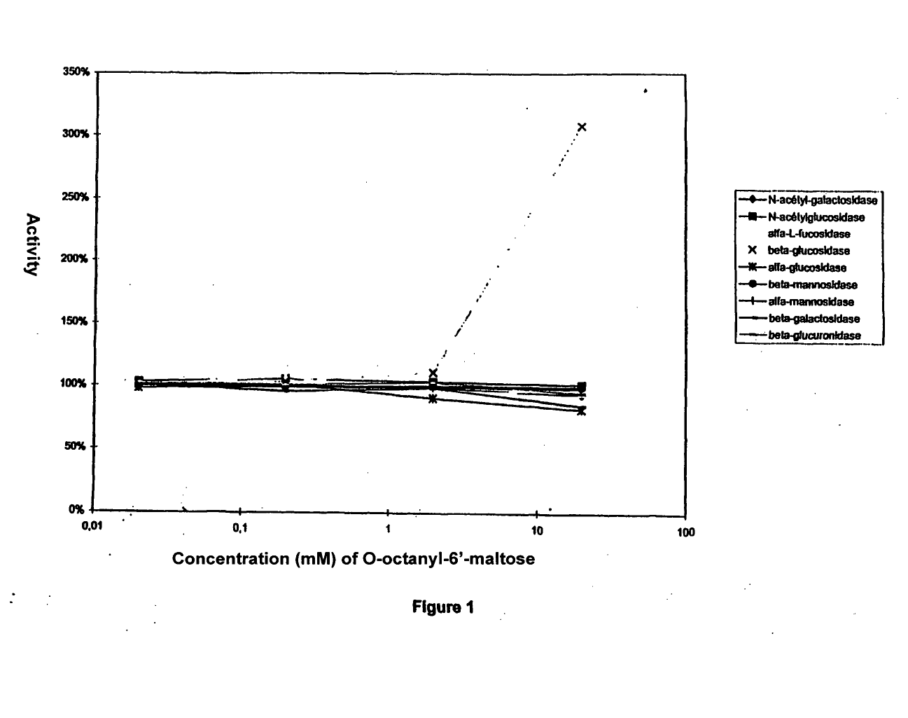

The results shown in FIG. 1 show that of the glycosidases tested, only β-glucosidase had kinetics influenced by O-octanoyl-6′-maltose. The other glycosidases were either indifferent to the presence of O-octanoyl-6′-maltose or very slightly inhibited. β-D-glucosidases (lysosomial) are involved in glycolipid catabolism and in particular, glucosyl-ceramides to ceramides. Thus, they are specifically involved in the barrier function.

### 1.2 Optimum Concentration of O-octanoyl-6′-maltose

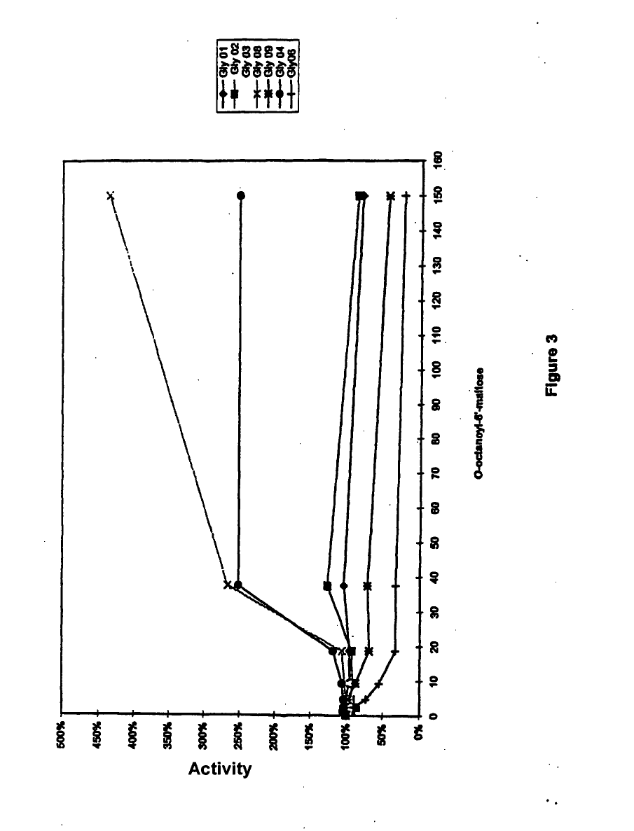

The optimum concentration of O-octanoyl-6′-maltose for the highest β-D-glucosidase activity was sought by comparing increasing product concentrations. The results shown in FIG. 3 show that maximum stimulation was observed for concentrations of about 40 mM (1%). This result shows that O-octanoyl-6′-maltose can be used in a concentration of about 1% in a cosmetic composition for stimulating β-glucosidase activity and thus improving the barrier fuction, said concentration being in a range that is cosmetically acceptable. Neither maltose alone nor glucose alone could stimulate β-D-glucosidase activity; However, O-octanoyl-6-D-glucose also revealed a certain stimulation of activity, albeit small, indicating that the carbohydrate-carbon chain combination is the factor that acts on glucosidase activity.

### 1.3 Effect of O-octanoyl-6-D-maltose on Thermostable Recombinant Glycosidases

O-octanoyl-6′-maltose was also tested for its effect on glycosidases sold by Clonezyme. Table 1 shows the substrate specificities of each glycosidase. FIG. 3 shows the results of 7 glycosidases tested in the presence of increasing concentrations of O-octanoyl-6′-maltose.

TABLE 1

| | GLY- | GLY- | GLY- | GLY- | GLY- | GLY- | GLY- | GLY- | GLY- | GLY- |
|---|---|---|---|---|---|---|---|---|---|---|
| | 01 | 02 | 03 | 04 | 05 | 06 | 07 | 08 | 09 | 10 |
| β-D-cellobiose | − | ++ | ε | ε | | + | + | ε | ε | − |
| β-D-galactose | − | ++ | ε | + | + | ε | ε | + | − | − |
| α-D-glucose | − | − | − | − | − | − | − | − | − | − |
| β-D-glucose | ++ | ++ | ++ | ++ | ++ | ++ | ++ | ++ | ++ | − |
| β-N-acetyl-D-glucosaminide | − | − | − | − | − | − | − | − | − | − |
| β-D-fucose | − | ++ | ++ | ++ | ++ | ++ | ++ | + | ++ | − |
| β-L-fucose | − | − | − | − | − | − | − | − | − | − |
| β-D-glucuronide | − | − | − | − | − | − | ε | − | − | − |
| α-D-galactose | − | − | − | − | − | − | − | − | − | + |
| β-D-mannose | − | ε | − | − | − | + | + | − | + | − |
| α-D-mannose | − | − | − | − | − | − | − | − | − | − |
| β-D-xylose | ε | + | − | ε | ε | ε | ε | ε | ε | − |
| α-L-arabinofuranoside | − | − | − | − | − | − | − | − | − | − |
| α-L-arabinopyranoside | − | + | ε | ε | ε | ε | ε | ε | ε | − |
| β-D-lactose | − | + | ε | − | − | + | ε | − | − | − |
| α-L-rhamnose | − | − | − | − | − | − | − | − | − | − |
| α-D-N-acetylneuramide | − | − | − | − | − | − | − | − | − | − |
| β-D-N-acetylchitobioside | − | − | − | − | − | − | − | − | − | − |
| α-L-fucose | − | − | − | − | − | − | − | − | − | − |

(−: no activity; ε, +, ++: specific activity)

The data shown in FIG. 3 show that not all of the test glycosidases were stimulated in the same manner in the presence of O-octanoyl-6′-maltose. A first group of enzymes was insensitive. A second group of enzymes was inhibited. A third group exhibited a stimulation of up to 400% of their base activity.

In conclusion, this study showed that:

1) carbohydrate derivatives specifically activate β-glucosidases, and in particular those present in the stratum corneum;

2) this effect is weaker or even absent or the opposite for other test glycosidases, suggesting a stimulating effect in these products, specific for β-glucosidases.

### 2 IN VIVO EFFECT OF O-OCTANOYL-6′-MALTOSE ON IMPROVING THE SKIN BARRIER EFFECT

The specific stimulation of O-octanoyl-6′-maltose on β-D-glucosidase activity suggests an effect of carbohydrate derivatives improving the skin barrier function. A study was carried out to verify this hypothesis in vivo.

The aim of the study was to evaluate the effect of O-octanoyl-6′-maltose on the barrier function, over 4 weeks.

### 2.1 Experimental Protocol

Experimental section: The study included 70 volunteers, all female and aged 18 to 50 years and having dry skin on their legs (score >2) and an insensible water loss, (IWL), measured in g/m².h, of more than 8.

For each individual, one leg was treated and one leg was untreated (left or right, selected randomly), constituting two statistical groups (treated, untreated).

The mean IWL was 10.79. The mean dryness score was 2.68.

Twice-daily application to the leg to be treated was carried out following right/left randomization. The barrier effect was evaluated using IWL (insensible water loss) and lag time (time in seconds for the appearance of redness due to methyl nicotinate).

In order to measure the insensible water loss (IWL), a Courage and Khasaka Tewameter was used, in accordance with the manufacturer's instructions.

In order to measure the lag time after applying methyl nicotinate, a Doppler Perimed laser was used in accordance with the manufacturer's instructions.

A statistical analysis of the data was then carried out:
the mean data at week 0 (TO) and at 4 weeks (T4) were compared using a Student t test for matched pairs;
the means of the treated zones and control zones at T4 were compared using a Student t test for matched pairs;
the different treatments were compared using a single factor variance analysis (the product). A Thukey test allowed multiple 2 by 2 comparisons of the means (treated against bare skin) at T4 (4 weeks);
the observed effects were quantified by the mean percentage change in the treated zone reduced by the mean percentage in the control zone.

### 2.2 Results

the means and standard deviations for the lag time and IWL parameters are indicated at T0 and T4, NS signifying a negative test result (not significant), S signifying a positive test result (significant). The treatments are shown in the first column:
Vehicle, composition comprising only the vehicle constituted by a standard mixture of:
   Oil, 12%
   Thickening agent (Colomer), 0.3%;
   Non ionic surfactants, 5% (PEG-SQ stearate (Myri), 2.5% and glyceryl stearate/PEG-100 stearate (Arlacel), 2.5%)
   Water, qsp 100%;
Bare skin, no treatment;
Carbohydrate, composition comprising 2.17% of O-octanoyl-6′-maltose [α-D-glucopyranosyl-1-4-D-glucopyranose].

Table 1 below shows the effect of different treatments at T0 and T4 and compares the lag time and IWL before and after treatment.

TABLE 1
Study as a function of time

| | Lag time | | | IWL | | |
|---|---|---|---|---|---|---|
| | T0 | Sig. | T4 | T0 | Sig | T4 |
| Vehicle | 215 ± 85 | S p < 0.001 | 391 ± 102 | 7.25 ± 1.17 | S p = 0.015 | 6.26 ± 1.51 |
| Bare skin | 230 ± 59 | S p < 0.001 | 374 ± 86 | 7.23 ± 1.55 | NS | 6.87 ± 2.19 |
| Carbohydrate | 243 ± 115 | S p < 0.001 | 448 ± 146 | 7.32 ± 1.59 | S p = 0.003 | 6.22 ± 0.86 |
| Bare skin | 248 ± 114 | S p = 0.016 | 389 ± 195 | 7.05 ± 1.62 | NS | 7.05 ± 1.41 |

Table 2 below compares the lag time and IWL parameters after each treatment against the no treatment control (bare skin).

TABLE 2
Study of treatment effects at 4 weeks

| | Lag time | IWL |
|---|---|---|
| Vehicle | 391 ± 102 | 6.26 ± 1.51 |
| Significance | NS | S p = 0.037 |
| Bare skin | 374 ± 86 | 6.87 ± 2.19 |
| Carbohydrates | 448 ± 146 | 6.22 ± 0.86 |
| Significance | NS | S p = 0.002 |
| Bare skin | 389 ± 195 | 7.05 ± 1.41 |

Table 3 shows the values of the effects observed for each treatment compared with the no-treatment control at T4.

TABLE 3
Comparison of treatments

| | Lag time | IWL |
|---|---|---|
| | T4_{treated} − T4_{control} | T4_{treated} − T4_{control} |
| Vehicle | 17 ± 80 | −0.61 ± 1.10 |
| Carbohydrate | 59 ± 168 | −0.83 ± 0.92 |

Table 4 below shows the percentage changes in the parameters for each treatment compared with the no-treatment control.

TABLE 4
Percentage change at 4 weeks

| | Lag time | IWL |
|---|---|---|
| Vehicle | 5% | −9% |
| Carbohydrate | 15% | −12% |

### 2.3 Discussion of Results

The results show that the vehicle and the “carbohydrate” treatment produced a significant reduction in IWL compared with bare skin (see Table 2).

It should be noted that the “carbohydrate” treatment significantly reduced the IWL (−12%) (see Table 4).

This result indicates an “in vivo” effect of O-octanoyl-6′-maltose on improving the skin barrier function, in particular via a significant reduction in IWL.

### 3 EXAMPLES OF FORMULATIONS

Composition 1: Face Milk

| Vaseline oil | 7.0 | g |
|---|---|---|
| O-octanoyl-6′-maltose | 1.0 | g |
| Glyceryl monostearate, polyethylene glycol stearate | 3.0 | g |
| (100 OE) | | |
| Carboxyvinyl polymer | 0.4 | g |
| Stearyl alcohol | 0.7 | g |
| Soya proteins | 3.0 | g |
| NaOH | 0.4 | g |
| Preservative | qs | |
| Water | qsp 100 | g |

This composition was in the form of a face milk with good cosmetic properties and was mild and comfortable in use.

The pH of the composition was about 5.5.

Composition 2: Lotion

| O-octanoyl-6′-maltose | 0.5 | g |
|---|---|---|
| 2-ethylhexyl palmitate | 10.0 | g |
| Cyclopentadimethylsiloxane | 20.0 | g |
| Butylene glycol | 5.0 | g |
| Preservative | qs | |
| Water | qsp 100 | g |

This lotion, which contained no surfactants, encouraged skin desquamation.

Composition 3: Milk

| Octyl palmitate | 35.0 | g |
|---|---|---|
| Glycerin | 2.0 | g |
| O-octanoyl-6′-maltose | 0.8 | g |
| C10-C30 acrylate/alkylacrylate cross-linked polymer | 0.1 | g |
| Triethanolamine | 0.1 | g |
| Wheat amino acids | 1.0 | g |
| Preservative | qs | |
| Water | qsp 100 | g |

The milk obtained, which contained no surfactants, had good cosmetic properties.

Composition 4: Face Gel

| Glycerin | 10.0 | g |
|---|---|---|
| O-octanoyl-6′-maltose | 1.0 | g |
| Disodium cocoamphodiacetate | 1.0 | g |
| Preservative | qs | |
| Water | qsp 100 | g |

The gel obtained had good cosmetic properties.

Composition 5: Water Cleansing Gel

| Butylene glycol | 7.0 | g |
|---|---|---|
| Sodium lauroyl sarcosinate | 4.0 | g |
| O-octanoyl-6′-maltose | 1.0 | g |
| Triethanolamine | 0.8 | g |
| Carbomer | 0.5 | g |
| Preservative | qs | |
| Water | qsp 100 | g |

The gel obtained had good cosmetic properties.

1-12. (canceled)

13. A method for cosmetic treatment of the skin to improve barrier function, comprising applying to the skin of a subject in need thereof a composition comprising at least one carbohydrate or carbohydrate derivative of formula (I):
R—X-A (I)
wherein A represents a chain composed of one to twenty carbohydrate units or carbohydrate derivatives each containing 3 to 6 carbon atoms, connected together, each of said carbohydrate units or derivatives optionally being substituted;
wherein R represents a linear or branched alkyl chain or an alkenyl chain containing 1 to 24 carbon atoms, which are optionally interrupted by ether bridges and which optionally carry a hydroxyl function, a carboxylic acid function, an amine function, an ester function, an acyloxy function, an amide function, an ether function, a carbamate function or a urea function; and
wherein X represents a function connecting R and A.

14. The method according to claim 13, wherein the carbohydrate units or derivatives are connected together via acetal bridges.

15. The method of claim 13, wherein each of said carbohydrate units or derivatives is optionally substituted with a halogen, an amine function, an acid function, an ester function, a thiol, an alkoxy function, a thio-ether function, a thio-ester function, an amide function, a carbamate function, or a urea function.

16. The method of claim 13, wherein R represents a linear or branched alkyl chain or an alkenyl chain containing 4 to 24 carbon atoms.

17. The method of claim 13, wherein X is an amine, ether, amide, ester, urea, carbamate, thioester, thioether, or sulphonamide function.

18. The method of claim 13, wherein the at least one carbohydrate or carbohydrate derivative is selected using an in vitro test that quantifies the stimulant effect of said derivative on the β-D-glucosidase activity, said test comprising the following steps:
a) producing a mixture comprising the carbohydrate derivative, a β-D-glucosidase, a chromogenic substrate for said β-D-glucosidase, and a suitable buffer solution;
b) quantifying the rate of the enzymatic β-glucosidase reaction by assaying the quantity of chromophores released by cleavage of the chromogenic substrate; and
c) selecting a carbohydrate or carbohydrate derivative for which the reaction rate is improved compared with the rate of reaction measured in a control solution in the absence of the carbohydrate or carbohydrate derivative.

19. The method of claim 13, wherein the carbohydrate derivative is O-octanoyl-6′-maltose.

20. The method of claim 13, wherein the at least one carbohydrate or carbohydrate derivative comprises 0.05% to 20% by weight of the total composition weight.

21. The method of claim 20, wherein the at least one carbohydrate or carbohydrate derivative comprises 0.2% to 10% by weight of the total composition weight.

22. The method of claim 21, wherein the at least one carbohydrate or carbohydrate derivative comprises 0.5% to 5% by weight of the total composition weight.

23. The method of claim 13, wherein the composition further comprises at least one complementary hydrophilic or lipophilic sunscreen which is active in the UVA and/or UVB region and which optionally comprises a sulphonic function.

24. The method of claim 13, wherein the composition is suitable for topical administration.

25. A composition for improving the skin barrier function, comprising at least one carbohydrate or carbohydrate derivative of formula (I):
R—X-A (I)
wherein A represents a chain composed of one to twenty carbohydrate units or carbohydrate derivatives each containing 3 to 6 carbon atoms, connected together, each of said carbohydrate units or derivatives optionally being substituted;
wherein R represents a linear or branched alkyl chain or an alkenyl chain containing 1 to 24 carbon atoms, which are optionally interrupted by ether bridges and which optionally carry a hydroxyl function, a carboxylic acid function, an amine function, an ester function, an acyloxy function, an amide function, an ether function, a carbamate function or a urea function; and
wherein X represents a function connecting R and A.

26. The composition of claim 25, wherein the carbohydrate is O-octanoyl-6′-maltose.

27. The composition of claim 25, wherein the composition is suitable for topical administration.

28. The method of claim 13, wherein the at least one carbohydrate or carbohydrate derivative is present in a β-glucosidase activity stimulating amount.
